(19) United States
(12) Patent Application Publication
(10) Pub. No.: US 2015/0327809 A1
(43) Pub. Date: Nov. 19, 2015

(54) BIOLOGICAL INFORMATION MEASURING DEVICE, MEASURING UNIT OF BIOLOGICAL INFORMATION MEASURING DEVICE, FINGER ACCOMMODATING UNIT OF BIOLOGICAL INFORMATION MEASURING DEVICE, AND PULSE OXYMETER

(71) Applicant: KONICA MINOLTA, INC., Chiyoda-ku, Tokyo (JP)

(72) Inventors: NORIHIRO TATEDA, SAKAI-SHI, OSAKA (JP); KENJI KAWADA, KAIZUKA-SHI, OSAKA (JP); NOBUAKI SHIGENAGA, SAKAI-SHI, OSAKA (JP)

(73) Assignee: KONICA MINOLTA, INC., TOKYO (JP)

(21) Appl. No.: 14/648,796

(22) PCT Filed: Nov. 21, 2013
(86) PCT No.: PCT/JP2013/081338

§ 371 Date: Jun. 1, 2015

(30) Foreign Application Priority Data
Dec. 6, 2012 (JP) 2012-267072

(51) Int. Cl. A61B 5/00; A61B 5/1455

(52) U.S. Cl. CPC A61B 5/6826; A61B 5/14552; A61B 5/6838

(57) ABSTRACT

A biological information measuring device includes: a finger accommodating unit to be attached to a finger of a living subject; and a measuring unit that can be connected to the finger accommodating unit, and includes a light emitting unit and a light receiving unit that face each other, with the region in which the finger is to be placed in the finger accommodating unit being interposed between the light emitting unit and the light receiving unit. After formed as components independent of each other, the finger accommodating unit and the measuring unit are connected. The basic electrical components (such as the light emitting unit, the light receiving unit, and an electrical circuit) related to the measuring function of the biological information measuring device are complete in the measuring unit.

### TECHNICAL FIELD

The present invention relates to a biological information measuring device such as a pulse oxymeter that is attached to a finger of a living subject and optically obtains biological information from the finger, a measuring unit of a biological information measuring device, and a finger accommodating unit of a biological information measuring device.

### BACKGROUND ART

Pulse oxymeter is known as a device to measure a degree of oxygen saturation in blood (SpO2). In this pulse oxymeter, a measuring unit attached to a body site of a subject calculates SpO2 based on the quantity of light that has passed through the body site or been reflected by the body site after light was emitted toward the body site.

As for this pulse oxymeter, there has been a suggested device in which a light source, a sensor, a process, an amplifier, and the like are placed in an integrated housing (see Patent Literature 1, for example). With this structure, the device manufacturing costs are lowered, and the device is not easily broken. There has been another suggested device in which a main unit is attached to a wrist, and a probe is secured to a finger with belt-like tape (see Patent Literature 2, for example).

### CITATION LIST

### Patent Literatures

Patent Literature 1: EP 1 830 695 B

Patent Literature 2: JP 2005-110816 A

### SUMMARY OF INVENTION

### Technical Problem

According to the technology disclosed in Patent Literature 1, however, a special metal mold is required in integrally molding the structure of the integrated housing, and it is difficult to determine the positions of a circuit substrate and the like. The size of the portion of the housing to be attached to a finger (also called the finger accommodating unit) is determined by the size of the special metal mold. Therefore, it is difficult to change only the size of the finger accommodating unit to a size for adults or a side for children, for example. In view of this, it is necessary to prepare special metal molds for respective sizes in the production of pulse oxymeters. However, product inventory control and distribution management are not easy.

Patent Literature 2 discloses a structure that adjusts the inner diameter of a ring-like locking portion in accordance with the size of a finger (or a structure that changes the size of the finger accommodating unit). By the technology disclosed in Patent Literature 2, however, long-time attachment of the device puts a burden on the subject, due to the main unit attached to the wrist and the cable extending from the main unit to the probe secured to the finger.

This problem occurs not only in a pulse oxymeter but also in any measuring device that is attached to a finger of a living subject and obtains biological information from the finger.

The present invention has been made in view of the above problem, and the first object thereof is to provide a biological information measuring device that can easily change its size in production and reduce the burden on the subject due to long-time attachment of the device to his/her finger.

The second object of the present invention is to make an improvement for efficiently supply biological information measuring devices while achieving the first object.

### Solution to Problem

In order to solve at least one of the above objects, a biological information measuring device, reflecting an aspect of the present invention, that obtains biological information about a living subject by receiving light emitted from a light emitting element with a light receiving element, with a finger of the living subject being inserted into a space between a pair of photoelectric elements formed with the light emitting element and the light receiving element, one of the light emitting element and the light receiving element being a first photoelectric element, the other one of the light emitting element and the light receiving element being a second photoelectric element, the biological information measuring device includes: (a) a measuring unit configured to optically measure the biological information, using the pair of the first photoelectric element and the second photoelectric element as the pair of photoelectric elements; and (b) a finger accommodating unit configured to hold the finger of the living subject, the finger accommodating unit being formed as a component independent of the measuring unit, the finger accommodating unit being connected to the measuring unit, wherein the measuring unit includes: a main unit including a housing configured to hold the first photoelectric element in an optically exposed position, and an internal electronic circuit connected to the first photoelectric element, the internal electronic circuit being placed inside the housing; and an arm configured to hold the second photoelectric element in such a position that the second photoelectric element optically faces the first photoelectric element with the finger accommodating unit being interposed in between, and hold a transmission path for power supply and signal transmission between the second photoelectric element and the internal electronic circuit, the arm being integrally formed with the main unit, and optical measurement of the biological information using the pair of photoelectric elements, and transmission of a result of the measurement to outside are performed by the internal electronic circuit.

In order to solve at least one of the above objects, a measuring unit of a biological information measuring device, reflecting an aspect of the present invention, that obtains biological information about a living subject by receiving light emitted from a light emitting element with a light receiving element, with a finger of the living subject being inserted into a space between a pair of photoelectric elements formed with the light emitting element and the light receiving element, one of the light emitting element and the light receiving element being a first photoelectric element, the other one of the light emitting element and the light receiving element being a second photoelectric element, the measuring unit includes: a main unit including a housing configured to hold the first photoelectric element in an optically exposed position, and an internal electronic circuit connected to the first photoelectric element, the internal electronic circuit being placed inside the housing; and an arm configured to hold the second photoelectric element in such a position that the second photoelectric element optically faces the first photoelectric element with a predetermined finger accommodating unit being interposed in between, and hold a transmission path for power supply and signal transmission between the second photoelectric element and the internal electronic circuit, the arm being integrally formed with the main unit, wherein the finger accommodating unit is configured to hold the finger of the living subject, the finger accommodating unit being formed as a component independent of the measuring unit and being connected to the measuring unit, and optical measurement of the biological information using the pair of photoelectric elements, and transmission of a result of the measurement to outside are performed by the internal electronic circuit.

In order to solve at least one of the above objects, a finger accommodating unit reflecting an aspect of the present invention holds a finger of a living subject in a biological information measuring device that obtains biological information about the living subject by receiving light emitted from a light emitting element with a light receiving element, with the finger of the living subject being inserted into a space between a pair of photoelectric elements formed with the light emitting element and the light receiving element, one of the light emitting element and the light receiving element being a first photoelectric element, the other one of the light emitting element and the light receiving element being a second photoelectric element, a measuring unit including: a main unit including a housing configured to hold the first photoelectric element in an optically exposed position, and an internal electronic circuit connected to the first photoelectric element, the internal electronic circuit being placed inside the housing; and an arm configured to hold the second photoelectric element in such a position that the second photoelectric element optically faces the first photoelectric element with the finger accommodating unit being interposed in between, and hold a transmission path for power supply and signal transmission between the second photoelectric element and the internal electronic circuit, the arm being integrally formed with the main unit, optical measurement of the biological information using the pair of photoelectric elements, and transmission of a result of the measurement to outside being performed by the internal electronic circuit, drive control on the pair of photoelectric elements, calculation and generation of a detection information signal based on a result of optical detection by the pair of photoelectric elements, and transmission of the detection information signal to the outside of the device are performed by the internal electronic circuit, the finger accommodating unit being formed in a ring-like shape as a component independent of the measuring unit, the finger accommodating unit having a connecting structure connectable to the measuring unit.

### Advantageous Effects of Invention

In a biological information measuring device reflecting an aspect of the present invention, the basic functions necessary for an optical measuring operation involving light emission and reception, and transmission of data obtained through the measuring operation are complete in the measuring unit. Therefore, the finger accommodating unit does not need to have any of those functions, and the finger accommodating unit can be readily manufactured at low costs.

Further, the measuring unit and the finger accommodating unit are formed as independent components, and are then connected. Accordingly, in a case where the finger accommodating unit comes in different sizes (such as a size for adults and a size for children), the manufacturer of the biological information measuring device manages the measuring unit and the finger accommodating unit as independent components, and can produce the biological information measuring device by connecting the measuring unit and the finger accommodating unit as necessary after receiving an order. That is, with the biological information measuring device of the present invention, it is possible to readily produce a biological information measuring device that fits the size of a finger of a user, and furthermore, product inventory control and distribution management are facilitated. As a result, biological information measuring devices can be efficiently supplied.

Further, the finger accommodating unit is connected to the measuring unit. Therefore, there is no need to attach one of the components of the measuring unit to a site at a distance from the finger (see JP 2005-110816 A, for example), and the cable or the like for connecting the measuring unit and the finger accommodating unit is not necessary. Accordingly, the burden on the subject due to the attachment of the biological information measuring device to the finger can be reduced.

The measuring unit of a biological information measuring device reflecting an aspect of the present invention can be used in the above described biological information measuring device.

The finger accommodating unit of a biological information measuring device reflecting an aspect of the present invention can be used in the above described biological information measuring device.

### BRIEF DESCRIPTION OF DRAWINGS

FIG. 1 is a schematic perspective view of the exterior of a biological information measuring device according to an embodiment.

FIG. 2 is a schematic side view of the exterior of the biological information measuring device according to the embodiment.

FIG. 3 is a schematic front view of the exterior of the biological information measuring device according to the embodiment.

FIG. 4 is a schematic top view of the structure of the biological information measuring device according to the embodiment.

FIG. 5 is an X-Z cross-sectional view taken along the position indicated by a dot-and-dash line III-III in FIG. 3.

FIG. 6 is a Y-Z cross-sectional view taken along the position indicated by a dot-and-dash line V-V in FIG. 4.

FIG. 7 is a diagram showing a situation before the finger accommodating unit is attached to the measuring unit in the embodiment.

FIG. 8 is a diagram showing a situation where the finger accommodating unit is attached to the measuring unit in the embodiment.

FIG. 9 is a block diagram showing the functional structure of the biological information measuring device according to the embodiment.

FIG. 10 is a block diagram showing the functional structure of the electrical circuit according to the embodiment.

FIG. 11 is a diagram showing a situation where the biological information measuring device according to the embodiment is attached to a finger.

FIG. 12 is a diagram schematically showing the structure of a biological information measuring device according to a first modification.

FIG. 13 is a diagram showing an example of attachment of the biological information measuring device according to the first modification to a finger.

FIG. 14 is a diagram showing a situation before the finger accommodating unit is attached to the measuring unit in a second modification.

FIG. 15 is a diagram showing a situation where the measuring unit and a band are attached to the finger accommodating unit in a third modification.

FIG. 16 is a diagram showing the internal structure of the biological information measuring device in the third modification.

FIG. 17 is a diagram showing an example of a situation before the finger accommodating unit is attached to the measuring unit in a fourth modification.

FIG. 18 is a diagram showing an example of a situation before the finger accommodating unit is attached to the measuring unit in the fourth modification.

FIG. 19 is a diagram showing an example of a situation before the finger accommodating unit is attached to the measuring unit in the fourth modification.

### DESCRIPTION OF EMBODIMENTS

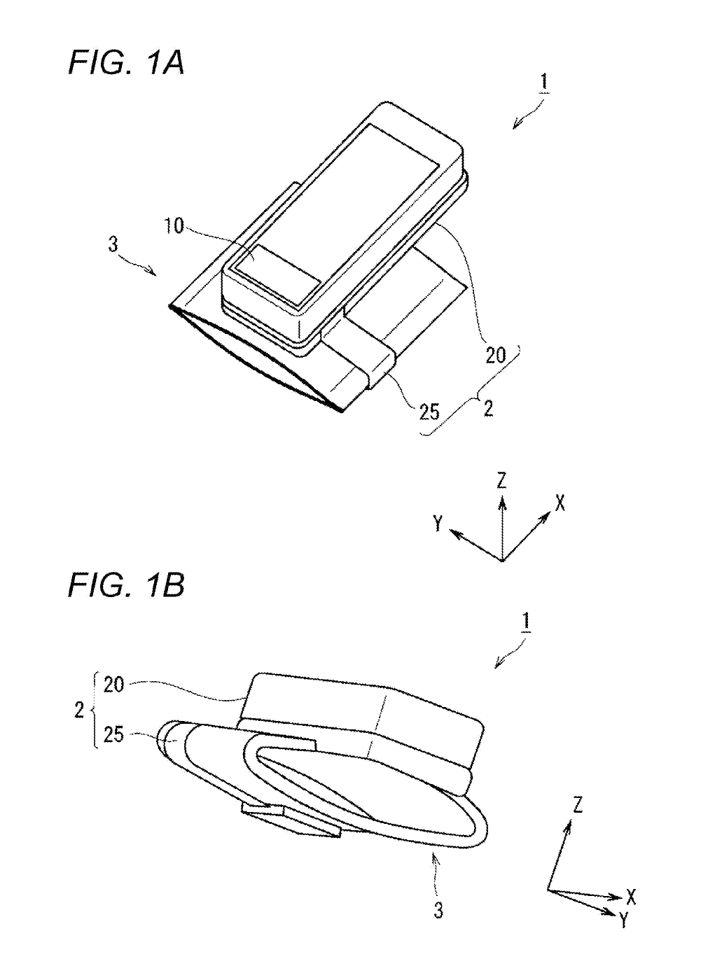

The following is a description of an embodiment of the present invention, with reference to the drawings. In the drawings, components having like structures and functions are denoted by like reference numerals, and explanation of them will not be repeated more than once. The drawings are schematic, and the sizes of the respective structures and the positional relations among them in the drawings are not accurately shown. Each of FIGS. 1 to 8 and FIGS. 11 to 19 is accompanied by an X-Y-Z coordinate system of a right-handed type, with one longitudinal direction of a biological information measuring device 1 (the rightward direction in FIG. 1) being the +X-direction.

### (1) Embodiment

### (1-1) Structure of the Biological Information Measuring Device 1

The biological information measuring device 1 according to an embodiment of the present invention is a pulse oxymeter that has a light receiving unit 5 (a light receiving element) to receive light that has been emitted from a light emitting unit 4 (a light emitting element) and passed through a finger of a test subject (typically, a subject such as a patient), and obtains a digital value (an SpO2 value) of the degree of oxygen saturation in the blood based on a signal output from the light receiving unit 5.

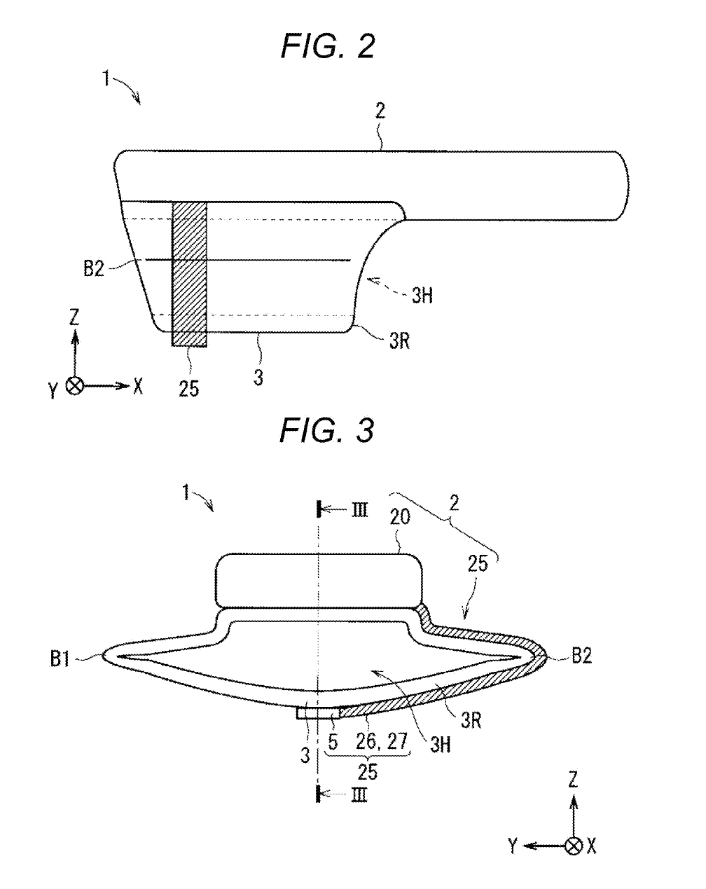

FIGS. 1 to 4 are diagrams schematically showing the exterior of the biological information measuring device 1. Of these drawings, FIG. 1 is a perspective view of the biological information measuring device 1. FIGS. 2, 3, and 4 are a side view, a front view, and a plan view of the biological information measuring device 1, respectively.

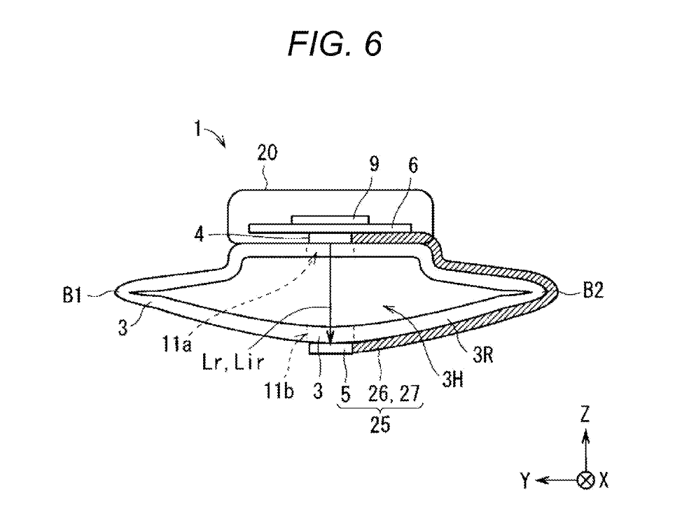

FIGS. 5 and 6 are diagrams schematically showing the structure of the biological information measuring device 1. FIG. 5 is an X-Z cross-sectional view taken along the dot-and-dash line III-III shown in FIG. 3. FIG. 6 is a Y-Z cross-sectional view taken along the dot-and-dash line V-V shown in FIG. 5.

As shown in the drawings, the biological information measuring device 1 mainly includes a measuring unit 2 and a finger accommodating unit 3. The measuring unit 2 is a structure in which a main unit 20 and an arm 25 are integrally formed, and the basic electrical components (the light emitting unit 4, the light receiving unit 5, an electrical circuit 6, and the like) related to the measuring function of the pulse oxymeter are complete in this measuring unit 2. The finger accommodating unit 3 has a structure that can hold a finger of a test subject (a subject). A mechanism for integrating the measuring unit 2 and the finger accommodating unit 3 is attached to the measuring unit 2 and/or the finger accommodating unit 3.

Therefore, a light emitting/receiving operation described later (a measuring operation through light emission and light reception) is performed in a situation where the measuring unit 2 and the finger accommodating unit 3 are connected and a finger of a subject is placed in the finger accommodating unit 3, so that biological information about the degree of oxygen saturation in the blood of the subject can be obtained.

The structure of the biological information measuring device 1 is described below in detail.

The main unit 20 includes a housing 20h and various kinds of components placed in the housing 20h. The various kinds of components placed in the housing 20h include the light emitting unit 4, the electrical circuit 6, a power supply unit 7, a charging unit 8, a communication unit 9, and an operating unit 10. The housing 20h has the shape of a rectangular parallelepiped, for example. If the housing 20h is harder than the finger accommodating unit 3, the various components placed in the main unit 20 are not easily damaged, and a finger of the subject is held by the finger accommodating unit 3 that is relatively soft. Accordingly, the biological information measuring device 1 can be comfortable to wear.

The arm 25 is a component integrally formed with the main unit 20, and includes the light receiving unit 5, a transmission path 26 that connects to the light receiving unit 5 and the electrical circuit 6 in such a manner as to electrically and optically transmit information, and a substrate 27 on which the light receiving unit 5 and the transmission path 26 are mounted. The substrate 27 may be a flexible substrate (hereinafter referred to as the “flexi-substrate 27”), for example. In this case, the flexi-substrate 27 is flexible and deformable, and the arm 25 is deformed as the finger accommodating unit 3 described later is deformed. Accordingly, the subject can feel comfortable wearing the biological information measuring device 1. The arm 25 preferably has an insulator or the like that covers the transmission path 26, the flexi-substrate 27, and the like, so as to prevent them from being exposed.

The light receiving unit 5 in the arm 25 is positioned so that the light emitting unit 4 and the light receiving unit 5 face each other, with the region in which a finger of a subject is to be placed being interposed between the light emitting unit 4 and the light receiving unit 5 in the finger accommodating unit 3. Therefore, in the light emitting/receiving operation, light emitted from the light emitting unit 4 passes through the finger of the subject, and is received by the light receiving unit 5. In this embodiment, the light emitting unit 4 is equivalent to the “first photoelectric element” in the present invention, the light receiving unit 5 is equivalent to the “second photoelectric element” in the present invention, and the pair of the light emitting unit 4 and the light receiving unit 5 is equivalent to the “pair of photoelectric elements” in the present invention.

The finger accommodating unit 3 is a component that is attached to a finger of the subject in the light emitting/receiving operation in the biological information measuring device 1 and fixes the positional relation between the finger and the measuring unit 2 in a relative manner. For example, if the finger accommodating unit 3 contains an elastic material that generates elastic force for holding a finger, it is easy to attach the biological information measuring device 1 to the finger. The elastic material may be a polymeric material such as rubber, a spring, or the like. Specifically, the entire finger accommodating unit 3 may be formed with a resin such as rubber having elasticity, or may be formed by burying a U-shaped flat spring in a resin, for example.

Further, a ring-like portion 3R forming an insertion hole 3H into which a finger of a living subject is to be inserted in the −X-direction is provided in the finger accommodating unit 3. Accordingly, the biological information measuring device 1 can be attached to a finger very easily, as the finger is inserted into the insertion hole 3H. The ring-like portion 3R is elastically deformed in the closing direction of the insertion hole 3H by virtue of the elastic force generated by the elastic material of the finger accommodating unit 3. Accordingly, the biological information measuring unit is stably attached to the finger, and the burden on the subject due to long-time attachment of the device to the finger can be reduced.

Although the finger accommodating unit 3 and the measuring unit 2 are formed independently of each other, the finger accommodating unit 3 and the measuring unit 2 are connected and used (see FIGS. 1 to 3, and FIGS. 5 and 6) when the light emitting/receiving operation is performed. That is, the mechanism for integrating the measuring unit 2 and the finger accommodating unit 3 is attached to the measuring unit 2 and/or the finger accommodating unit 3.

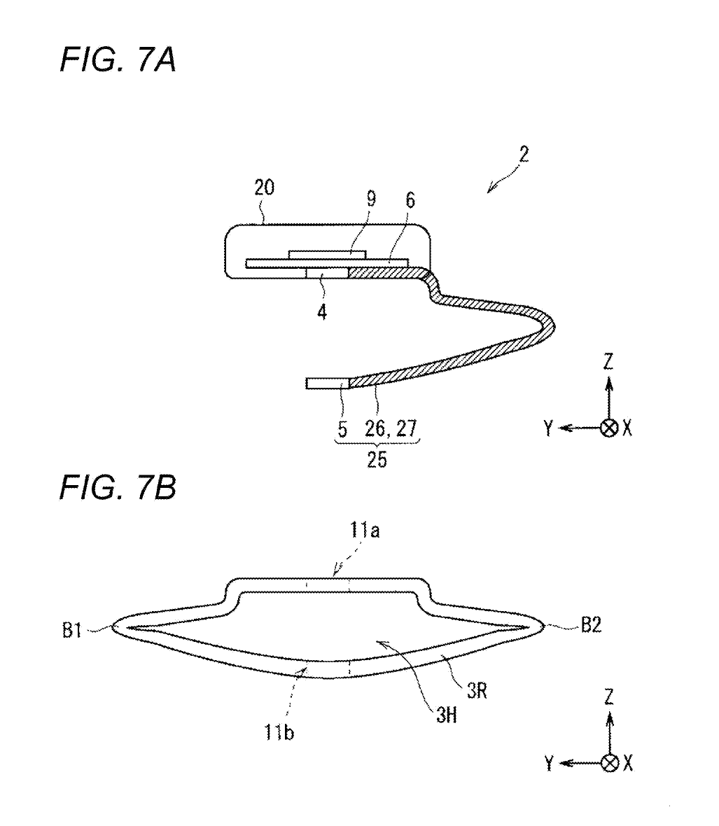

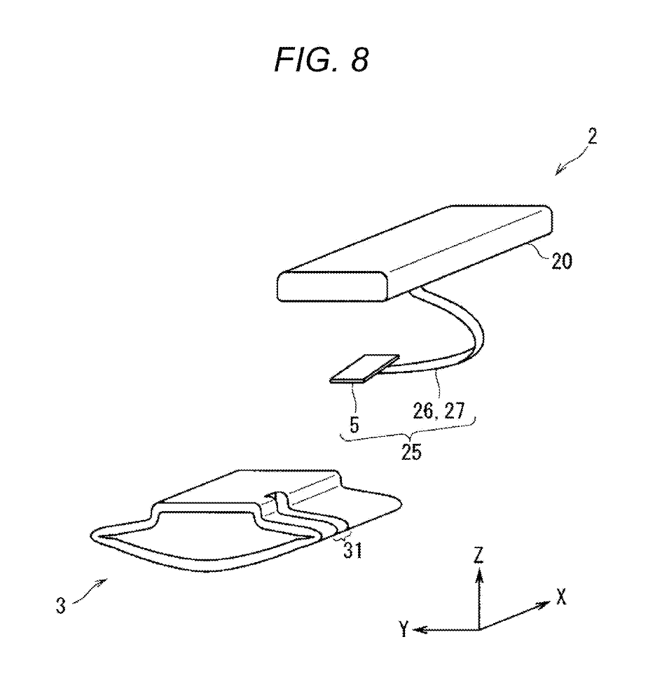

FIG. 7 is a diagram showing a situation prior to the finger accommodating unit 3 is attached to the measuring unit 2 in the biological information measuring device 1 (or a situation where the measuring unit 2 and the finger accommodating unit 3 are formed independently of each other). FIG. 8 is a diagram showing an example of a situation where the independent measuring unit 2 and the independent finger accommodating unit 3 are integrated. In this specification, the expression “the measuring unit 2 and the finger accommodating unit 3 are integrated” or “connected” means a situation where “the measuring unit 2 and the finger accommodating unit 3 are in contact with each other, and the finger accommodating unit 3 is not easily detached from the measuring unit 2 or where the positional relation between the measuring unit 2 and the finger accommodating unit 3 is not greatly changed”, and is not limited to a situation where “the measuring unit 2 and the finger accommodating unit 3 are completely secured to each other so as not to separate from each other”.

As shown in FIG. 7, and FIG. 8, in this embodiment, a groove 31 to be engaged with the arm 25 is formed along the outer circumference of the finger accommodating unit 3. Accordingly, when the arm 25 is engaged with the groove 31 formed on the finger accommodating unit 3, the measuring unit 2 and the finger accommodating unit 3 are connected and integrated. Further, if the groove 31 is filled with an adhesive agent or the like, and the arm 25 is then engaged with the groove 31, the measuring unit 2 and the finger accommodating unit 3 are bonded (secured) to each other with the adhesive agent, and are more stably connected. Particularly, detachment of the finger accommodating unit 3 from the measuring unit 2 can be effectively prevented in this case.

As described above, in the biological information measuring device 1 of this embodiment, the measuring unit 2 and the finger accommodating unit 3 are formed independently of each other, and are connected after the formation. Accordingly, in a case where the finger accommodating unit 3 can come in different sizes (such as a size for adults and a size for children), the manufacturer of the biological information measuring device 1 forms the respective components independently of each other, and can connect the measuring unit 2 and the finger accommodating unit 3 in accordance with a received order or secure the measuring unit 2 and the finger accommodating unit 3 to each other after the connection, to complete the biological information measuring device 1. As the last procedures in the manufacture can be carried out only when necessary as described above, product inventory control and distribution management can be facilitated.

In a case where finger accommodating units 3 of different sizes are manufactured, each of the finger accommodating units 3 of the different sizes needs to have the mechanism (the groove 31, for example) for connecting to the measuring unit 2. An example of such finger accommodating units 3 may be finger accommodating units 3 that have the outer shapes (such as the grooves 31) of the same size, but have different inner diameters. In this case, a user can use a finger accommodating unit 3 having the inner diameter that matches the size of a finger of the user. In another example, it is possible to use finger accommodating units 3 that are the same in the external shape (such as the groove 31) and the inner diameter, but differ in the elastic force. In this case, a user can use a finger accommodating unit 3 having the elastic force that matches the size of a finger of the user.

In the biological information measuring device 1 of this embodiment, the basic electrical components related to the measuring function of the pulse oxymeter are complete in the measuring unit 2, and accordingly, the finger accommodating unit 3 does not need to include any electrical component for measurement. The advantageous effects of this will be described later.

In the structure of the finger accommodating unit 3, a light emitting window 11a and a light receiving window 11b made of a material (such as glass) through which light easily passes are formed in the surface in contact with the light emitting unit 4 and the light receiving unit 5 (see FIG. 6 and FIG. 7). The reason for the provision of light emitting window 11a and the light receiving window 11b will be described later in conjunction with a detailed description of a measuring operation of the biological information measuring device 1.

### (1-2) Functional Structure of the Biological Information Measuring Device

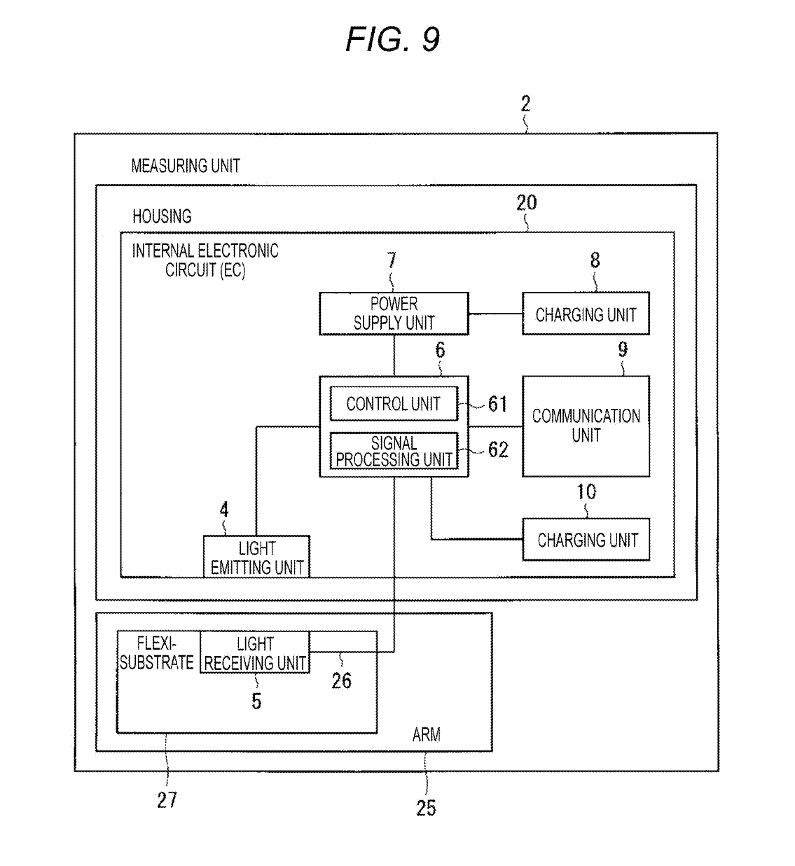

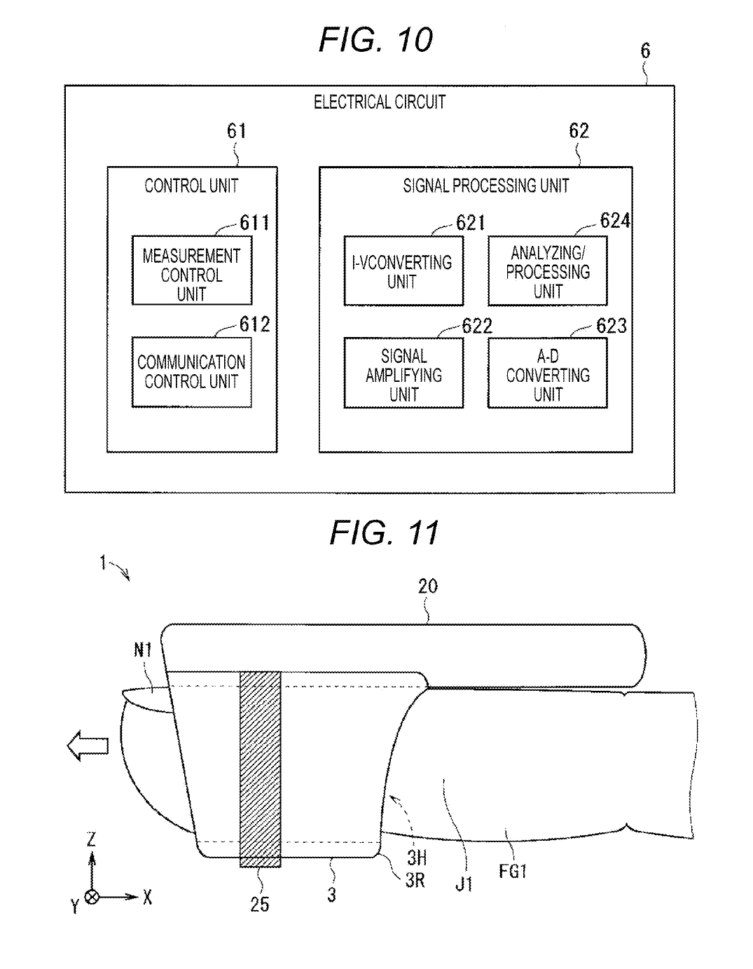

FIGS. 9 and 10 are block diagrams showing the functional structure of the biological information measuring device 1.

As shown in FIG. 9, the biological information measuring device 1 includes, as electrical components, the light emitting unit 4, the light receiving unit 5, the electrical circuit 6, the power supply unit 7, the charging unit 8, the communication unit 9, the operating unit 10, the transmission path 26, and the flexi-substrate 27. Particularly, the electrical components EC formed with the light emitting unit 4, the electrical circuit 6, the power supply unit 7, the charging unit 8, the communication unit 9, and the operating unit 10 are the electrical components related to the main unit 20, and are equivalent to the “internal electronic circuit” in the present invention.

The light emitting unit 4 is electrically connected to the electrical circuit 6. This light emitting unit 4 emits light to the light receiving unit, when supplied with power from the power supply unit 7 under the control of the electrical circuit 6. In FIGS. 5 and 6, the path in which the light travels (the light path) is indicated by arrows. The light emitting unit 4 includes a portion that emits light of the wavelength λ1 of the red region, and a portion that emits light of the wavelength λ2 of infrared rays. As the light emitting unit 4, an LED (Light Emitting Diode) or the like can be used, for example. At the time of measurement, repetitive pulse light emission is performed so that red light Lr of the wavelength λ1 and infrared light Lir of the wavelength λ2 are emitted from the light emitting unit 4 alternately in terms of time.

The light receiving unit 5 is electrically connected to the electrical circuit 6 via the transmission path 26 mounted on the flexi-substrate 27. This light receiving unit 5 outputs a current signal of the magnitude corresponding to the intensity of received light, to a signal processing unit 62 that will be described later. The light receiving unit 5 includes a photoelectric conversion element such as a silicon photodiode that is sensitive at least to light of the wavelength λ1 and light of the wavelength λ2. With a finger being inserted into the insertion hole 3H, the light receiving unit 5 receives light of the wavelengths λ1 and λ2 that has been emitted from the light emitting unit 4 and has passed through the body tissues of the finger.

At the time of measurement of biological information, the red light Lr of the wavelength λ1 and the infrared light Lir of the wavelength λ2 are emitted from the light emitting unit 4 alternately in terms of time, and a light receiving operation is performed in the light receiving unit 5 in synchronization with the light emitting operation of the light emitting unit 4. The light emitting operation of the light emitting unit 4 and the light receiving operation of the light receiving unit 5 can be controlled by a control unit 61 that will be described later. The light emitting/receiving operation for the light Lr and Lir is repeated at intervals of 1/100 (seconds) to 1/30 (seconds), for example.

If the light emitting unit 4 is placed in the main unit 20 as in this embodiment, the wiring path for supplying power to the light emitting unit 4 can be short. Accordingly, influence of noise on the electrical circuit 6 and the like due to the power supply to the light emitting unit 4 can be reduced.

The electrical circuit 6 includes the control unit 61 and the signal processing unit 62. This electrical circuit 6 may be formed with various electronic components, integrated circuit components, a CPU, and the like. Further, as shown in FIG. 10, the control unit 61 includes a measurement control unit 611, a communication control unit 612, and a charging-unit control unit (not shown). The signal processing unit 62 includes a current-to-voltage converting unit (hereinafter referred to as the I-V converting unit) 621, a signal amplifying unit 622, an analog-to-digital converting unit (hereinafter referred to as the A-D converting unit) 623, and an analyzing/processing unit 624.

The measurement control unit 611 controls operation of the light emitting unit 4 and the light receiving unit 5. Here, the light emitting unit 4 is caused to emit the red light Lr of the wavelength λ1 and the infrared light Lir of the wavelength λ2 alternately in terms of time at intervals of 1/100 (seconds), for example. The communication control unit 612 controls data communication being performed by the communication unit 9, which will be described later.

The I-V converting unit 621 converts a current signal that is periodically output from the light receiving unit 5, into a voltage signal. This voltage signal is a signal related to analog pulse waves (also referred to as the pulse wave signal).

The signal amplifying unit 622 is an amplifier that amplifies the voltage signal that is output from the I-V converting unit 621, for example.

The A-D converting unit 623 converts the analog pulse wave signal that is output from the signal amplifying unit 622, into a digital pulse wave signal. As a result, a digital value related to the pulse waves is obtained. That is, a digital value related to the pulse waves is obtained based on the current signal output from the light receiving unit 5 having received light that has been emitted from the light emitting unit 4 and has passed through a finger.

The analyzing/processing unit 624 conducts a predetermined data analysis based on the digital pulse wave signal output from the A-D converting unit 623. As a result, the following various values are calculated: the light quantities of the light Lr and Lir received by the light receiving unit 5, the amplitude of the pulse waves, the ratio between the amplitude of the red light Lr and the amplitude of the infrared light Lir, the value of the degree of oxygen saturation in the blood (the SpO2 value), the pulse rate, the pulse intervals (periods), and the like.

The measurement control unit 611, the communication control unit 612, and the analyzing/processing unit 624 may be formed with special-purpose electronic circuits, or may be realized by a microprocessor, a DSP (Digital Signal Processor), or the like executing a program. The I-V converting unit 621, the signal amplifying unit 622, and the A-D converting unit 623 may be formed with special-purpose electronic circuits, for example.

The power supply unit 7 includes a secondary battery such as a nickel hydrogen storage battery or a lithium ion battery. Power is supplied from the power supply unit 7 to the respective components such as the light emitting unit 4 and the electrical circuit 6 in the biological information measuring device 1.

The charging unit 8 is a circuit for charging the secondary battery in the power supply unit 7. For example, a charger may be connected to a terminal electrically connected to the secondary battery so that the secondary battery is charged. Accordingly, the secondary battery can be charged with a simple structure. In a case where the charging unit 8 performs non-contact charging on the secondary battery or where the charging unit 8 has a circuit for performing non-contact charging on the secondary battery, for example, the terminal or the like to which a charger or the like is to be connected is not necessary. Therefore, the secondary battery can be charged with an even simpler structure. As a non-contact charging method, a method that utilizes the electromagnetic induction of a coil or the like can be employed, for example. Therefore, the main unit 2 does not need to include the mechanism (such as a lid that can be opened and closed) for exchanging primary batteries such as dry-cell batteries. As a result, the structure of the main unit 20 that is simple but is not easily broken can be realized.

The communication unit 9 transmits data acquired by the signal processing unit 62 to an external device such as a monitoring (display) device or a therapeutic apparatus in a wireless manner. Therefore, the biological information measuring device 1 does not need to include the structure for analyzing and saving signals transmitted from the biological information measuring device 1, and a display unit that displays measurement results. As a result, the biological information measuring device can be made smaller in size, power consumption can be reduced, and manufacturing costs can be lowered.

Further, as wireless data communication is performed in this embodiment, the burden on the subject (such as a cable getting in the subject's way) due to attachment of the biological information measuring device 1 to the subject can be made lighter than in a case where cable data communication is performed (or where the main unit 20 and an external device are connected with the cable, for example).

The digital pulse wave signal obtained by the A-D converting unit 623 of the signal processing unit 62, or the digital value data of the pulse waves, may be transmitted by the communication unit 9, for example. In this case, various values are calculated by a structure equivalent to the analyzing/processing unit 624 in the external device (such as a personal computer) that has received the data transmitted from the communication unit 9, for example. In this manner, the structure for processing signals in the biological information measuring device 1 can be simplified. Accordingly, the device can be made smaller in size, power consumption can be reduced, and the manufacturing costs can be further lowered.

Here is an assumed case where the signal processing unit 62 obtains at least one of the digital values related to the value of the degree of oxygen saturation in the blood (the SpO2 value), the pulse rate, and the pulse intervals (periods) based on the digital pulse signal. In this case, at least one of the digital values related to the value of the degree of oxygen saturation in the blood (the SpO2 value), the pulse rate, and the pulse intervals (periods), which is obtained by the signal processing unit 62, may be transmitted by the communication unit 9. Accordingly, useful information can be readily obtained by an external device that has received the data transmitted from the communication unit 9, without any special arithmetic operation or the like performed in the external device. Further, the display unit that displays measurement result does not need to be included in the biological information measuring device 1. Accordingly, the device can be made smaller in size, power consumption can be reduced, and the manufacturing costs can be lowered.

The electrical circuit 6 may include a memory of some sort that stores data obtained by the signal processing unit 62. In that case, the biological information measuring device 1 is attached to a person in an outdoor medical crisis for which neither a monitoring device nor a therapeutic apparatus can be prepared, for example. Measurement is then carried out, and measurement data is stored into the memory. After the emergency patient is transported to a hospital or an ambulance or the like, the measurement data is read from the memory, and is transmitted to a monitoring device and a therapeutic apparatus, so that the conditions of the patient immediately after the problem suddenly occurred can be retraced in terms of time. In this manner, the data can be used with a time lag.

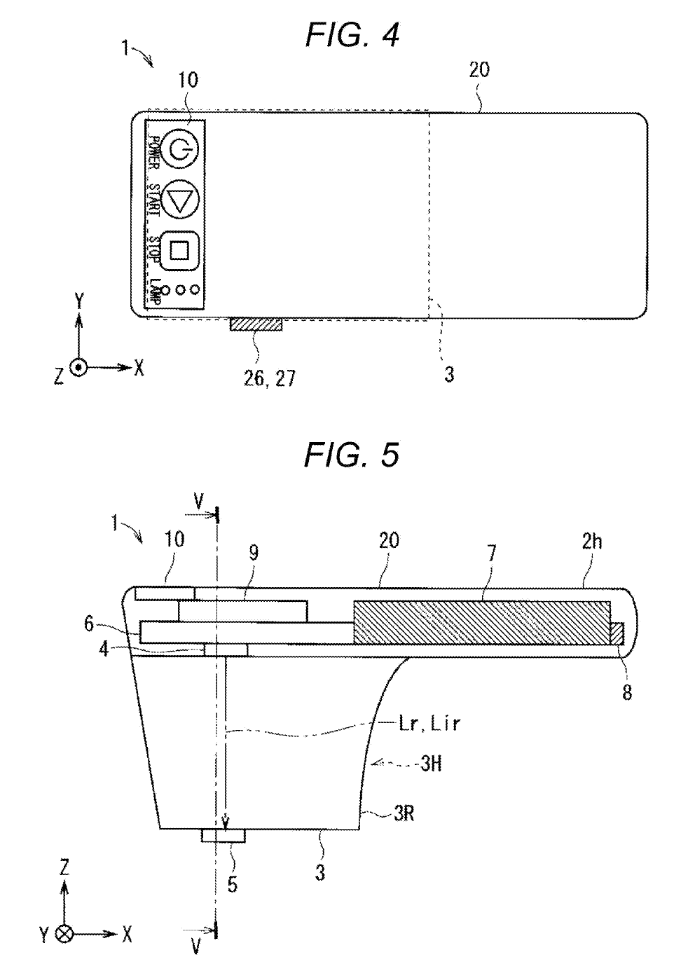

The operating unit 10 includes a power button, a measurement start button, and a measurement end button, for example (see FIG. 4). The power button is the button for switching on and off the power supply from the power supply unit 7 to the respective components of the biological information measuring device 1. The measurement start button is the button for starting measurement of the value of the degree of oxygen saturation in the blood (the SpO2 value) or the like. The measurement end button is the button for ending measurement of the value of the degree of oxygen saturation in the blood (the SpO2 value).

### (1-3) Attachment of the Biological Information Measuring Device to a Finger

FIG. 11 shows an example of a form of the biological information measuring device 1 in a situation where a finger FG1 is inserted into the insertion hole 3H.

In a situation where the biological information measuring device 1 is not attached to a finger as shown in FIGS. 2 and 3, for example, the elastic force for holding the finger inserted into the insertion hole 3H is generated from the elastic material of the finger accommodating unit 3, and the ring-like portion 3R is elastically deformed in the direction in which the insertion hole 3H is deformed to a flat rhombus, closing in the Z-direction. At this point, the ring-like portion 3R may be folded at portions B1 and B2 located on the ±Y-sides, as shown in FIG. 3, for example.

When the biological information measuring device 1 is attached to a finger, on the other hand, a user (a medical staff member or the test subject) manually performs an operation to elastically deform the ring-like portion 3R so as to widen the insertion hole 3H in the Z-direction, in spite of the elastic force generated from the elastic material of the finger accommodating unit 3, and insert the finger FG1 into the insertion hole 3H in the −X-direction as shown in FIG. 11. Accordingly, in the situation where the biological information measuring device 1 is attached to the finger FG1, the ring-like portion 3R is deformed in the direction in which the insertion hole 3H is closed in the Z-direction by virtue of the elastic force generated by the elastic material of the finger accommodating unit 3. As a result, the finger FG1 is held by the finger accommodating unit 3. At this point, for example, the finger FG1 is inserted into the insertion hole 3H in such a manner that the region between the nail N1 and the distal interphalangeal joint (also called the first joint) J1 of the finger FG1 inserted into the insertion hole 3H is irradiated with the light Lr and Lir emitted from the light emitting unit 4.

The above described light emitting/receiving operation (the measuring operation) is performed in a situation where a finger of the subject is inserted into the finger accommodating unit 3 as shown in FIG. 11. The light Lr and Lir emitted from the light emitting unit 4 passes through the finger accommodating unit 3 and the finger FG1 of the subject, and is received by the light receiving unit 5. Therefore, the light emitting window 11a and the light receiving window 11b made of a material (such as glass) through which the light Lr and Lir can easily pass are formed in the region of the finger accommodating unit 3 (the surface of the finger accommodating unit 3 in contact with the light emitting unit 4 or the light receiving unit 5) through which the light Lr and Lir can pass. Accordingly, the light emitting unit 4 and the light receiving unit 5 are optically exposed, and the accuracy of measurement to be carried out by the biological information measuring device 1 becomes higher.

### (1-4) Summary of the Embodiment

As described above, in the biological information measuring device 1 according to this embodiment, the basic electrical components (such as the light emitting unit 4, the light receiving unit 5, and the electrical circuit 6) related to the measuring function of the pulse oxymeter are complete in the measuring unit 2. Therefore, the finger accommodating unit 3 does not need to have any of the functions of those components, and serves as a mechanism that can hold a finger of a living subject and is integrated with the measuring unit 2. Accordingly, the finger accommodating unit 3 can have a simpler structure and be manufactured at low costs.

Further, in a case where the finger accommodating unit comes in different sizes (such as a size for adults and a size for children), for example, the measuring unit 2 and the finger accommodating unit 3 are managed as independent components. After an order is received, the measuring unit 2 and the finger accommodating unit 3 are bonded and firmly secured to each other with an adhesive agent or the like, so that the biological information measuring device 1 can be produced. That is, in the biological information measuring device 1 of this embodiment, the size of the finger accommodating unit 3 can be easily set in accordance with the size of a finger of a subject. Further, product inventory control and distribution management are facilitated.

In a case where the finger accommodating unit 3 includes one of the basic electrical components related to the measuring function of the pulse oxymeter (or the group of electrical components that realize the functions necessary to achieve the original objective of measurement in this device), no tests can be conducted on the measuring function of the biological information measuring device 1 before the finger accommodating unit 3 is connected to the measuring unit 2. This is because the light receiving unit or the light emitting unit has not been assembled yet. However, where all the basic electrical components necessary for measurement are included in the measuring unit 2 according to a feature of the present invention, a test can be advantageously conducted on the measuring function of the biological information measuring device 1 before the finger accommodating unit 3 is connected to the measuring unit 2.

As the finger accommodating unit 3 is attached directly to the measuring unit 2, there is no need to attach part of the measuring unit 2 (such as the main unit 20) to a site other than a finger. Therefore, the cable or the like that connects the main unit and the finger accommodating unit in the oxymeter disclosed in JP 2005-118016 A is not necessary. Accordingly, the burden on the subject due to attachment of the biological information measuring device 1 to a finger can be reduced.

### (2) Modifications

It should be noted that the present invention is not limited to the above described embodiment, and various modifications and improvements can be made to the embodiment, without departing from the scope of the present invention.

### (2-1) First Modification

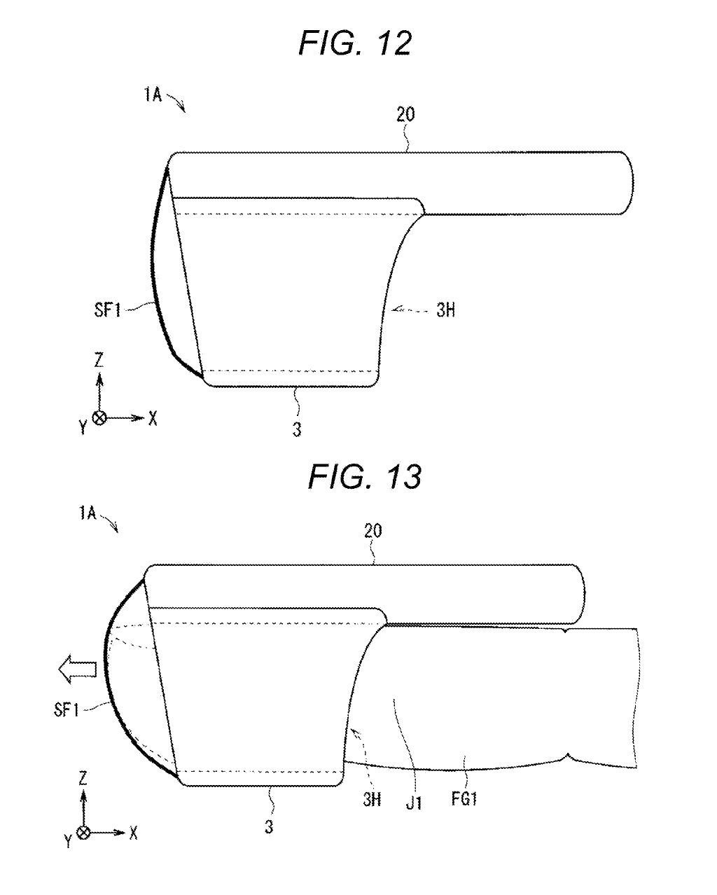

Although the insertion hole 3H is open in the ±X-directions in the biological information measuring device 1 according to the above described embodiment, the present invention is not limited to that. For example, it is possible to use a biological information measuring device 1A that has a stopper portion SF1 at one end of the insertion hole 3H of the finger accommodating unit 3 in the −X-direction as shown in FIG. 12. In this case, a finger FG1 is inserted from the +X-side into the insertion hole 3H as shown in FIG. 13, and the finger FG1 is brought into contact with the stopper portion SF1. Through this operation, the biological information measuring device 1A can be swiftly and appropriately attached to the portion of the finger FG1 to be irradiated with light. That is, the device position can be easily determined when the biological information measuring device 1A is attached to the finger FG1.

If the stopper portion SF1 is made of a material having elasticity, such as rubber, the finger is not easily injured when the finger FG1 is inserted into the insertion hole 3H, and accordingly, the biological information measuring device 1A can be easily attached to the tip of the finger. Further, the stopper portion SF1 easily fits the tip of the finger. Accordingly, the burden on the subject due to long-time attachment of the biological information measuring device 1A to the finger can be reduced. Further, if the stopper portion SF1 is made of a light blocking material that blocks light transmission, the light receiving unit 5 is not easily irradiated with external light by virtue of the light blocking effect. Accordingly, a measurement error is not easily caused.

### (2-2) Second Modification

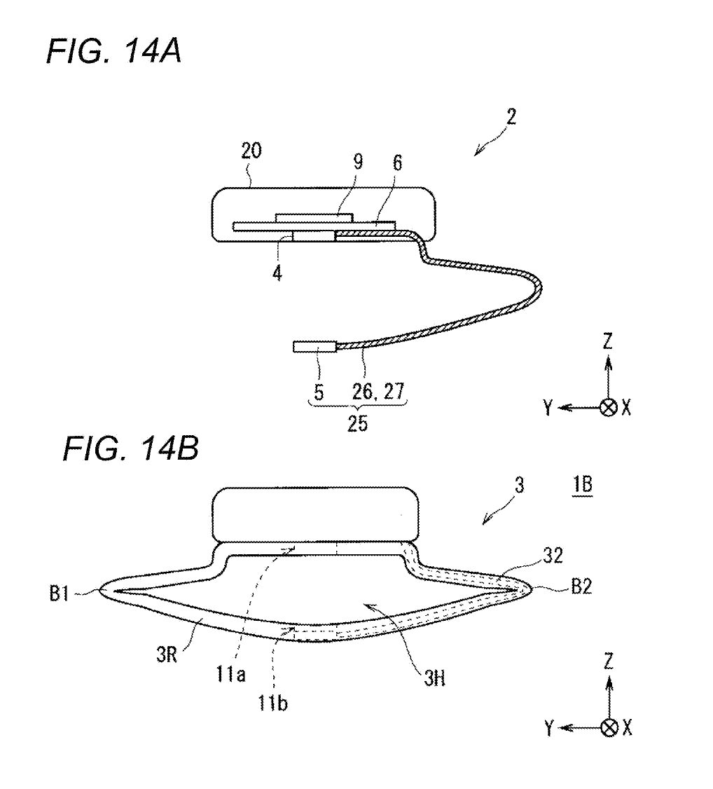

Although the arm 25 is engaged with the groove 31 formed along the outer circumference of the finger accommodating unit 3 so as to integrate the finger accommodating unit 3 with the measuring unit 2 in the biological information measuring device 1 according to the above described embodiment, the present invention is not limited to that. For example, as in a biological information measuring device 1B shown in FIGS. 14(a) and 14(b), it is possible to employ a structure (a tunnel structure) in which an insertion hole 32 into which the arm 25 is to be inserted is formed in the finger accommodating unit 3. In this case, the arm 25 (the light receiving unit 5, the transmission path 26, the flexi-substrate 27, and the like) is inserted into the insertion hole 32, so that the measuring unit 2 and the finger accommodating unit 3 are connected. As a result, the electrical components (such as the light receiving unit 5, the transmission path 26, and the flexi-substrate 27) of the arm 25 are protected inside the finger accommodating unit 3. Accordingly, these electrical components are not easily broken, compared with the electrical components in a case where the arm 25 is attached to an external portion of the finger accommodating unit 3 as in the above described embodiment.

### (2-3) Third Modification

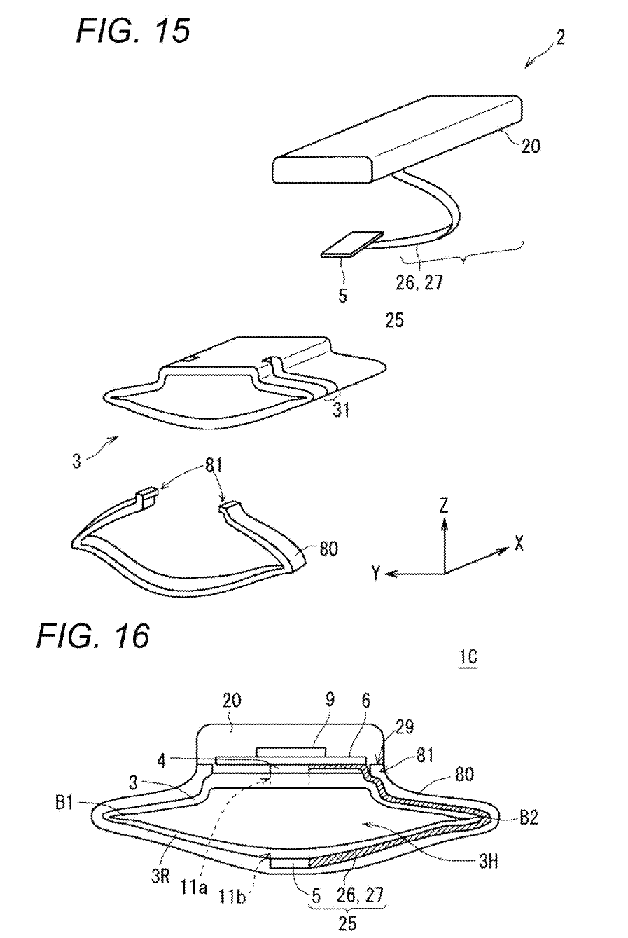

As another embodiment, it is possible to employ a biological information measuring device 1C that includes not only the components of the biological information measuring device 1 according to the above described embodiment, but also a band 80 that covers the engagement portion between the arm 25 and the groove 31 (FIGS. 15 and 16). As the electrical components (such as the light receiving unit 5, the transmission path 26, and the flexi-substrate 27) of the arm 25 are protected inside the band 80, these electrical components are not easily broken, compared with the electrical components in a case where the arm 25 is attached to an external portion of the finger accommodating unit 3 as in the above described embodiment. The band 80 preferably has insulating properties.

Particularly, in this modification, the groove 31 is formed along a U-shaped portion on the outer circumference minus an upper portion in a Y-Z plan view of the finger accommodating unit 3, and the U-shaped band 80 is engaged with the groove 31 in the Y-Z plan view. Accordingly, the adhesion between the band 80 and the finger accommodating unit 3 is high. If the engagement portion between the band 80 and the groove 31 is secured with an adhesive agent or the like, the adhesion between the band 80 and the finger accommodating unit 3 becomes even higher, and the band 80 is not easily detached from the finger accommodating unit 3. In this modification, the arm 25, which is part of the measuring unit 2, is sandwiched between the band 80 and the finger accommodating unit 3. Therefore, as the adhesion between the band 80 and the finger accommodating unit 3 becomes higher, the adhesion between the finger accommodating unit 3 and the measuring unit 2 becomes higher, and the finger accommodating unit 3 is not easily detached from the measuring unit 2.

In this modification, both end portions 81 of the band 80 in a U-shape are engaged with concave portions 29 formed in the lower surface of the main unit 20. If the two end portions 81 of the band 80 are secured to the concave portions 29 in the lower surface of the main unit 20 with an adhesive agent or the like, the adhesion between the band 80 and the main unit 20 becomes even higher, and the band 80 is not easily detached from the main unit 20.

Further, the structure of the biological information measuring device 1C of this embodiment that has the band 80 covering the engagement portion between the arm 25 and the groove 31 is easier to assemble than the structure of the biological information measuring device 1B of the second modification having the above described tunnel structure.

### (2-4) Fourth Modification

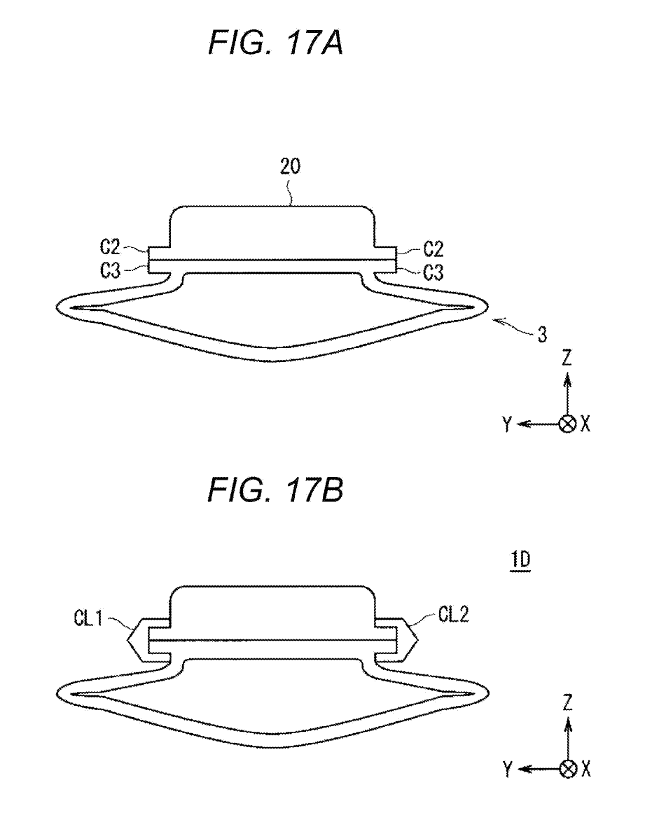

Although the method of firmly connecting the measuring unit 2 and the finger accommodating unit 3 is a securing method using an adhesive agent in the biological information measuring device 1 according to the above described embodiment, the present invention is not limited to that. FIGS. 17 to 19 are diagrams showing methods of securing the measuring unit 2 and the finger accommodating unit 3 to each other, which are different from the securing method using an adhesive agent explained in the above described embodiment. The components not related to the securing method (such as the arm 25, the light emitting window 11a, and the light receiving window 11b) are not shown.

As in a biological information measuring device 1D shown in FIGS. 17(a) and 17(b), it is possible to employ a structure in which clipped portions C2 and C3 are added to the measuring unit 2 and the finger accommodating unit 3, for example. In this case, the clipped portions C2 and C3 are in contact with each other, and are then clipped by clips CL1 and CL2. As a result, the measuring unit 2 and the finger accommodating unit 3 are secured to each other. The clipped portions C2 and C3, and the clips CL1 and CL2 are equivalent to the “clip engagement structure” in the present invention.

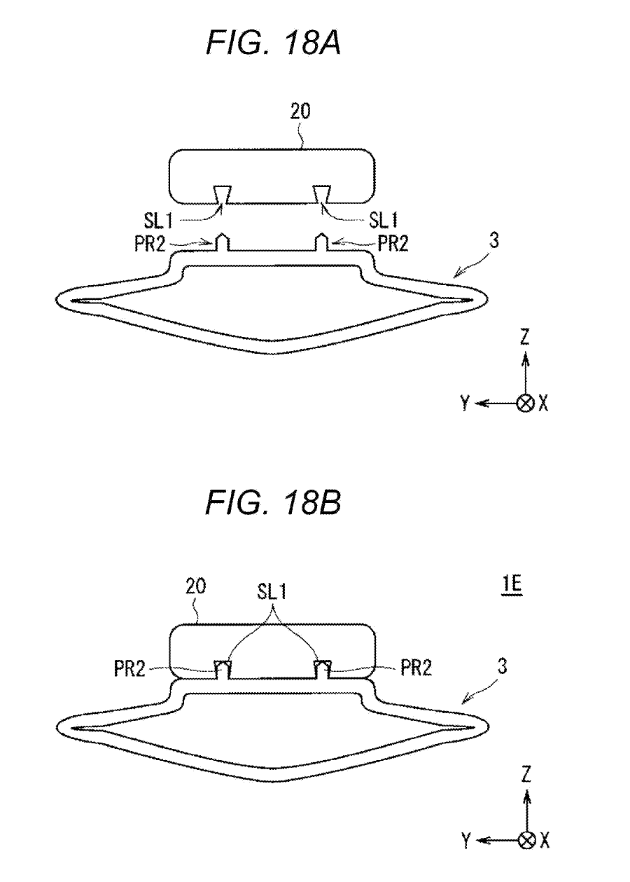

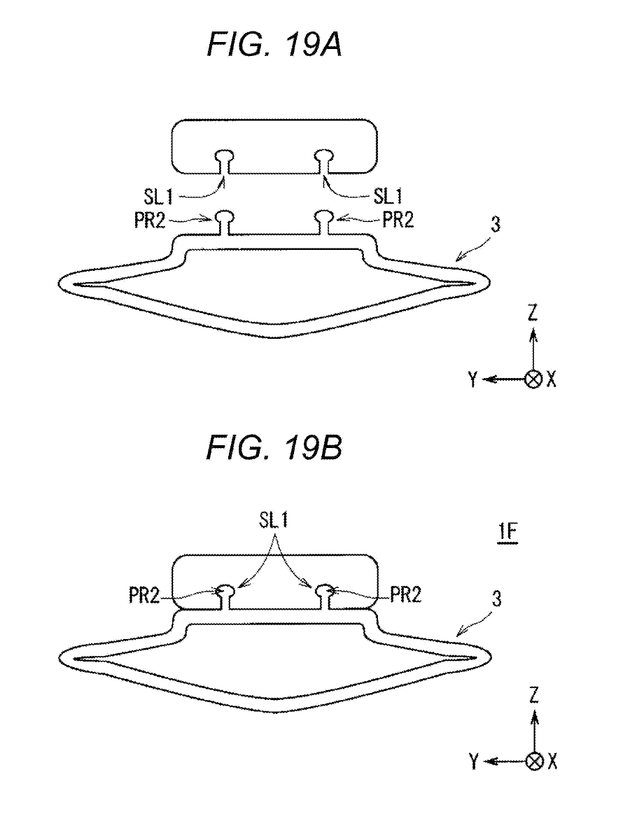

Further, as in a biological information measuring device 1E shown in FIGS. 18(a) and 18(b), it is possible to employ a structure in which protruding portions PR2 are formed on the finger accommodating unit 3, and slit portions SL1 corresponding to the protruding portions PR2 are formed in the main unit 20 of the measuring unit 2, for example. In this case, the protruding portions PR2 are pushed into the slit portions SL1, so that the measuring unit 2 and the finger accommodating unit 3 are secured to each other. Further, in a case where locking portions are formed inside the slit portions SL1 and at the tips of the protruding portions PR2 as in a biological information measuring device 1F shown in FIGS. 19(a) and 19(b), the measuring unit 2 and the finger accommodating unit 3 are more firmly secured to each other. The protruding portions PR2 and the slit portions SL1 are equivalent to the “concavity-convexity engagement structure” in the present invention. Alternatively, protruding portions may be added to the main unit 20 of the measuring unit 2, and slit portions may be formed in the finger accommodating unit 3, which is the opposite of this modification.

That is, other than the firm connecting method by which the groove 31 is filled with an adhesive agent and the measuring unit 2 and the finger accommodating unit 3 are secured to each other as in the above described embodiment, it is possible to employ a method of connecting the measuring unit 2 and the finger accommodating unit 3 in a structural manner as shown in FIGS. 17 to 19.

By this connecting method (see FIGS. 17 to 19), the measuring unit 2 and the finger accommodating unit 3 can be detachably connected. Further, if both the method of detachable connection and the firm connecting (securing) method using an adhesive agent are employed, the measuring unit 2 and the finger accommodating unit 3 are more firmly secured to each other.

In the former connecting method (the method of detachable connection), not only the manufacturer of the biological information measuring device 1 but also a user can connect the measuring unit 2 and the finger accommodating unit 3 or disconnect the measuring unit 2 and the finger accommodating unit 3 from each other. Accordingly, in a case where a user has finger accommodating units 3 of various sizes, for example, a suitable finger accommodating unit 3 can be selected as necessary before the biological information measuring device 1 is used. In a case where the finger accommodating unit 3 deteriorates or is damaged, a user can exchange the finger accommodating unit 3 for another finger accommodating unit 3 of the same size, instead of a finger accommodating unit 3 of a different size. Further, the finger accommodating unit 3 can be detached from the measuring unit 2 and be washed, and the washed finger accommodating unit 3 can be reattached to the measuring unit 2, for example. In a case where the method of detachably connecting the measuring unit 2 and the finger accommodating unit 3 is employed, there are various utilization forms as described above, and accordingly, the measuring unit 2 and the finger accommodating unit 3 can be efficiently used.

### (2-5) Fifth Modification

Although the arm 25 includes the light receiving unit 5, the transmission path 26, and the flexi-substrate 27 on which the light receiving unit 5 and the transmission path 26 are mounted in the biological information measuring device 1 according to the above described embodiment, the present invention is not limited to that. The requirements in the structure of the arm 25 are being integrally formed with the main unit 20, holding the light receiving unit 5 in such a manner that the light emitting unit 4 and the light receiving unit 5 optically face each other, and including the transmission path 26 that electrically connects the main unit 20 and the light receiving unit 5. Various kinds of arms 25 that satisfy these requirements can be employed.

Accordingly, it is possible to employ a structure that includes a substrate having the light receiving unit 5 mounted thereon, instead of the flexi-substrate 27 having the light receiving unit 5 and the transmission path 26 mounted thereon (a component of the above described embodiment), a holding unit that holds the substrate in such a manner that the light emitting unit 4 and the light receiving unit 5 face each other, and the transmission path 26 (such as a lead wire) that electrically connects the substrate and the main unit 20, for example. In another example, it is possible to employ a structure that includes a substrate having the light receiving unit 5 mounted thereon, the transmission path 26 that electrically connects the substrate and the main unit 20, and a flexible substrate having the transmission path 26 mounted thereon.

Further, the entire arm 25 has flexibility so that the finger accommodating unit 3 can be deformed and the biological information measuring device 1 becomes easy to wear, as described above. However, the present invention is not limited to that. That is, flexibility is not a requirement in the structure of the arm 25. Therefore, in a structure in which the arm 25 has a rigid housing that protects the above described electrical components (such as the light receiving unit 5, the transmission path 26, and the flexi-substrate 27), and has rigidity as a whole, for example, the biological information measuring device 1 is not easier to wear than the structure of the above described embodiment, but the shock resistance and the water resistance of the biological information measuring device 1 can be improved.

### (2-6) Other Modifications

In the biological information measuring device 1 according to the above described embodiment, the light emitting unit 4 is placed on the side of the main unit 20 (the +Z-side), and the light receiving unit 5 is placed on the flexi-substrate 27 (the −Z-side) so as to optically face the light emitting unit 4. However, the present invention is not limited to that. For example, it is possible to employ a structure in which the light receiving unit 5 is placed on the side of the main unit 20 (the +Z-side), and the light emitting unit 4 is placed on the flexi-substrate 27 (the −Z-side) so as to optically face the light receiving unit 5.

In this case, the light receiving unit 5 is equivalent to the “first photoelectric element” in the present invention, and the light emitting unit 4 is equivalent to the “second photoelectric element” in the present invention.

Although any display unit is not included in the biological information measuring device 1 according to the above described embodiment, the present invention is not limited to that. For example, a display unit for displaying various values obtained by the analyzing/processing unit 624 may be included. In this case, the communication control unit 612 and the communication unit 9 may not be included.

Although the finger accommodating unit 3 includes the ring-like portion 3R into which the finger FG1 is to be inserted in the biological information measuring device 1 according to the above described embodiment, the present invention is not limited to that. For example, the finger accommodating unit 3 may include components that sandwich the finger FG1. However, so as to reduce the burden on the subject due to long-time attachment of the biological information measuring device 1 to the finger, the finger accommodating unit 3 preferably includes the ring-like portion 3R into which the finger FG1 is to be inserted.

Although the finger accommodating unit 3 of the above described embodiment is made of an elastic material that generates an elastic force in the closing direction of the insertion hole 3H, the present invention is not limited to that. That is, the finger accommodating unit 3 may be made of a material that does not have elasticity (for example, a material that is not deformed and forms a round insertion hole 3H). In this case, however, different types of finger accommodating units 3 are preferably produced for each size of the insertion hole 3H so as to cope with the sizes of fingers of subjects.

Although the digital value of the degree of oxygen saturation in the blood (the SpO2 value) is obtained by the signal processing unit 62 in the biological information measuring device 1 according to the above described embodiment, the present invention is not limited to that. For example, it is possible to employ a biological information measuring device that is not a pulse oxymeter, does not obtain the SpO2 value, and measures biological information about pulse waves such as a pulse rate.

Although the I-V converting unit 621 is included in the electrical circuit 6 in the biological information measuring device 1 according to the above described embodiment, the present invention is not limited to that. For example, the I-V converting unit 621 that converts a current signal output from the light receiving unit 5 into an analog voltage signal may be mounted on the flexi-substrate 27. In this case, the transmission path of the current signal from the light receiving unit 5 to the I-V converting unit 621 becomes shorter, and the influence of noise can be reduced accordingly.

Further, for signal transmission in the biological information measuring device 1, not only an electrical signal transmission path but also an optical signal transmission path using an optical fiber or the like may be used.

The finger accommodating unit 3 including an electronic component not related to the basic electrical functions for measurement falls within the scope of the invention. Examples of such electronic components include an IC tag and an HF tag for identifying the type of the finger accommodating unit 3 that comes in various sizes. That is, the concept of the present invention lies in the point that the basic functions for measurement of biological information, which are the functions for

(1) optical measurement of the biological information with a pair of photoelectric elements, and

(2) transmission of results of the measurement to outside,

are complete with the electrical components in the measuring unit 2. The above tags and the like are not related to these basic functions.

The present invention can be applied not only in cases where the living subject is a human (a subject), but also to a device that obtains biological information about an animal from a finger of the animal at an animal hospital or livestock production facilities.

It is of course possible to combine all or some of the components of the above described embodiment and the respective modifications as appropriate without causing any contradiction.

### REFERENCE SIGNS LIST

1, 1A to 1F Biological information measuring device
2 Measuring unit
3 Finger accommodating unit
3H Insertion hole
3R Ring-like portion
4 Light emitting unit
5 Light receiving unit
6 Electrical circuit
7 Power supply unit
8 Charging unit
9 Communication unit
10 Operating unit
11a Light emitting window
11b Light receiving window
20 Main unit
20h Housing unit
25 Arm
26 Transmission path
27 Flexi-substrate
61 Control unit
62, 62D, 62E Signal processing unit
80 Band
621 Current-to-voltage converting unit (I-V converting unit)
622 Signal amplifying unit
623 Analog-to-digital converting unit (A-D converting unit)
624 Analyzing/processing unit
C1, C2 Clipped portion
CL1, CL2 Clip
F1 Flexible printed board
FG1 Finger
SF1 Stopper portion
SL1 Slit portion
PR2 Protruding portion

1. A biological information measuring device that obtains biological information about a living subject by receiving light emitted from a light emitting element with a light receiving element, with a finger of the living subject being inserted into a space between a pair of photoelectric elements formed with the light emitting element and the light receiving element,
one of the light emitting element and the light receiving element being a first photoelectric element, the other one of the light emitting element and the light receiving element being a second photoelectric element,
the biological information measuring device comprising:
(a) a measuring unit configured to optically measure the biological information, using the pair of the first photoelectric element and the second photoelectric element as the pair of photoelectric elements; and
(b) a finger accommodating unit configured to hold the finger of the living subject, the finger accommodating unit being formed as a component independent of the measuring unit, the finger accommodating unit being connected to the measuring unit,
wherein
the measuring unit includes:
a main unit including a housing configured to hold the first photoelectric element in an optically exposed position, and an internal electronic circuit connected to the first photoelectric element, the internal electronic circuit being placed inside the housing; and
an arm configured to hold the second photoelectric element in such a position that the second photoelectric element optically faces the first photoelectric element with the finger accommodating unit being interposed in between, and hold a transmission path for power supply and signal transmission between the second photoelectric element and the internal electronic circuit, the arm being integrally formed with the main unit, and
optical measurement of the biological information using the pair of photoelectric elements, and transmission of a result of the measurement to outside are performed by the internal electronic circuit.

2. The biological information measuring device according to claim 1, wherein
a groove structure matching a shape of the arm is formed in the finger accommodating unit, and
the measuring unit and the finger accommodating unit are connected, as the arm is engaged with the groove structure.

3. The biological information measuring device according to claim 1, wherein
a tunnel structure matching a shape of the arm is formed in the finger accommodating unit, and
the measuring unit and the finger accommodating unit are connected, as the arm is inserted into the tunnel structure.

4. The biological information measuring device according to claim 1, wherein the measuring unit and the finger accommodating unit are secured to each other with an adhesive agent, and are firmly connected.

5. The biological information measuring device according to claim 1, wherein the measuring unit and the finger accommodating unit are attachable/detachable from each other.

6. The biological information measuring device according to claim 5, wherein
a convex portion is formed on one of the main unit of the measuring unit and the finger accommodating unit, and a concave portion is formed on the other one of the main unit of the measuring unit and the finger accommodating unit, to form a concavity-convexity engagement structure in which the convex portion and the concave portion can be engaged with each other, and
the measuring unit and the finger accommodating unit are made attachable/detachable from each other by the concavity-convexity engagement structure.

7. The biological information measuring device according to claim 5, wherein
a clip engagement structure configured to hold the measuring unit and the finger accommodating unit in a pinching manner is formed, and
the measuring unit and the finger accommodating unit are made attachable/detachable from each other by the clip engagement structure.

8. The biological information measuring device according to claim 1, wherein the second photoelectric element and the transmission path in the arm are mounted on a flexible substrate.

9. The biological information measuring device according to claim 1, wherein the finger accommodating unit contains an elastic material generating an elastic force for holding the finger of the living subject.

10. The biological information measuring device according to claim 9, wherein
the finger accommodating unit includes a ring-like portion having an insertion hole into which the finger is to be inserted in one direction, and
the ring-like portion is elastically deformed in a closing direction of the insertion hole by the elastic force.

11. A measuring unit of a biological information measuring device that obtains biological information about a living subject by receiving light emitted from a light emitting element with a light receiving element, with a finger of the living subject being inserted into a space between a pair of photoelectric elements formed with the light emitting element and the light receiving element,
one of the light emitting element and the light receiving element being a first photoelectric element, the other one of the light emitting element and the light receiving element being a second photoelectric element,
the measuring unit comprising:
a main unit including a housing configured to hold the first photoelectric element in an optically exposed position, and an internal electronic circuit connected to the first photoelectric element, the internal electronic circuit being placed inside the housing; and
an arm configured to hold the second photoelectric element in such a position that the second photoelectric element optically faces the first photoelectric element with a predetermined finger accommodating unit being interposed in between, and hold a transmission path for power supply and signal transmission between the second photoelectric element and the internal electronic circuit, the arm being integrally formed with the main unit,
wherein
the finger accommodating unit is configured to hold the finger of the living subject, the finger accommodating unit being formed as a component independent of the measuring unit and being connected to the measuring unit, and
optical measurement of the biological information using the pair of photoelectric elements, and transmission of a result of the measurement to outside are performed by the internal electronic circuit.

12. A finger accommodating unit that holds a finger of a living subject in a biological information measuring device that obtains biological information about the living subject by receiving light emitted from a light emitting element with a light receiving element, with the finger of the living subject being inserted into a space between a pair of photoelectric elements formed with the light emitting element and the light receiving element,
one of the light emitting element and the light receiving element being a first photoelectric element, the other one of the light emitting element and the light receiving element being a second photoelectric element,
a measuring unit including:
a main unit including a housing configured to hold the first photoelectric element in an optically exposed position, and an internal electronic circuit connected to the first photoelectric element, the internal electronic circuit being placed inside the housing; and
an arm configured to hold the second photoelectric element in such a position that the second photoelectric element optically faces the first photoelectric element with the finger accommodating unit being interposed in between, and hold a transmission path for power supply and signal transmission between the second photoelectric element and the internal electronic circuit, the arm being integrally formed with the main unit,
optical measurement of the biological information using the pair of photoelectric elements, and transmission of a result of the measurement to outside being performed by the internal electronic circuit,
drive control on the pair of photoelectric elements, calculation and generation of a detection information signal based on a result of optical detection by the pair of photoelectric elements, and transmission of the detection information signal to the outside of the device are performed by the internal electronic circuit,
the finger accommodating unit being formed in a ring-like shape as a component independent of the measuring unit, the finger accommodating unit having a connecting structure connectable to the measuring unit.

13. A pulse oxymeter formed with the biological information measuring device according to claim 1,
the pulse oximeter measuring at least a degree of oxygen saturation in blood of the living subject as the biological information.

14. The biological information measuring device according to claim 2, wherein the measuring unit and the finger accommodating unit are secured to each other with an adhesive agent, and are firmly connected.

15. The biological information measuring device according to claim 2, wherein the measuring unit and the finger accommodating unit are attachable/detachable from each other.

16. The biological information measuring device according to claim 2, wherein the second photoelectric element and the transmission path in the arm are mounted on a flexible substrate.

17. The biological information measuring device according to claim 2, wherein the finger accommodating unit contains an elastic material generating an elastic force for holding the finger of the living subject.

18. A pulse oxymeter formed with the biological information measuring device according to claim 2,
the pulse oximeter measuring at least a degree of oxygen saturation in blood of the living subject as the biological information.

19. The biological information measuring device according to claim 3, wherein the measuring unit and the finger accommodating unit are secured to each other with an adhesive agent, and are firmly connected.

20. The biological information measuring device according to claim 3, wherein the measuring unit and the finger accommodating unit are attachable/detachable from each other.
